(19) United States
(12) Patent Application Publication
(10) Pub. No.: US 2021/0346566 A1
(43) Pub. Date: Nov. 11, 2021

(54) DISINFECTION APPARATUS AND METHOD OF TREATING WASTE

(71) Applicants: SCG Chemicals Co., Ltd., Bangkok (TH); INC Square, Co., Ltd., Pathumthani (TH)

(72) Inventors: Attawut Kumkrong, Bangkok (TH); Atitaya Panuvatvanich, Pathumthani (TH); Thammarat Koottatep, Chiangmai (TH); Sompong Hirunmasuwan, Pathumthani (TH); Pantaporn Sittplangkoon, Pathumthani (TH); Araya Wicheansan, Pichit (TH); Sakkarin Meephon, Kalasin (TH)

(73) Assignees: SCG Chemicals Co., Ltd., Bangkok (TH); INC Square, Co., Ltd., Pathumthani (TH)

(21) Appl. No.: 17/270,718

(22) PCT Filed: Aug. 22, 2019
(86) PCT No.: PCT/TH2019/000033

§ 371 Date: Feb. 23, 2021

(30) Foreign Application Priority Data
Aug. 24, 2018 (SG) 10201807200Q

(51) Int. Cl. A61L 11/00; A61L 2/04; A61L 2/24; B09B 3/00

(52) U.S. Cl. CPC A61L 11/00; A61L 2/04; A61L 2202/20; B09B 3/0083; A61L 2202/14; A61L 2/24

(57) ABSTRACT

A disinfection apparatus for treating waste may include a housing having an inlet and an outlet; a conveying mechanism which is disposed within the housing and which extends at least substantially along the housing from the inlet to the outlet; and a heating mechanism configured to transfer heat to the housing so as to heat an internal space of the housing to create a heated environment within the housing. A method of treating waste may include providing the waste in a heated environment within a housing of a disinfection apparatus; and conveying the waste, via a conveying mechanism of the disinfection apparatus, along a length of the housing of the disinfection apparatus. According to various embodiments, the method may further include heating, via a heating mechanism of the disinfection apparatus, the housing so as to heat an internal space of the housing to create the heated environment within the housing.

### TECHNICAL FIELD

Various embodiments generally relate to a disinfection apparatus and a method of treating waste. In particular, various embodiments generally relate to a disinfection apparatus for treating waste.

### BACKGROUND

Sewage or waste from a toilet or a lavatory and/or waste from livestock farm generally contains a mixture of solids (such as faeces) and liquid (such as urine and/or flush water). If the faeces are separated from the liquid and collected effectively from the sewage or the waste, the faeces can be used in agriculture application such as fertilizer. However, faeces collected from the toilet or the lavatory or the livestock farm generally contain high moisture content as well as total coliform bacteria such as faecal coliform. Accordingly, such faeces may not be suitable for direct use as fertilizer upon collection. This is because the high moisture content may not be suitable for direct agriculture application and the faecal coliform can be harmful to the environment and public health if the faeces are not treated to remove the faecal coliform before being use as fertilizer.

Accordingly, there is still a need for a disinfection apparatus for treating waste and a method of treating waste that addresses at least some of the issues identified above.

### SUMMARY

According to various embodiments, there is provided a disinfection apparatus for treating waste. The disinfection apparatus may include a housing having an inlet and an outlet. The disinfection apparatus may further include a conveying mechanism which is disposed within the housing and which extends at least substantially along a length of the housing from the inlet to the outlet. The disinfection apparatus may further include a heating mechanism configured to transfer heat to the housing so as to heat an internal space of the housing to create a heated environment within the housing.

According to various embodiments, there is provided a method of treating waste. The method may include providing the waste in a heated environment within a housing of a disinfection apparatus. The method may further include conveying the waste, via a conveying mechanism of the disinfection apparatus, along a length of the housing of the disinfection apparatus. According to various embodiments, the method may further include heating, via a heating mechanism of the disinfection apparatus, the housing so as to heat an internal space of the housing to create the heated environment within the housing.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1 shows a schematic diagram of a disinfection apparatus according to various embodiments;

FIG. 2 shows a schematic diagram of a disinfection apparatus according to various embodiments;

FIG. 3 shows a method of treating waste according to various embodiments;

FIG. 4 shows a temperature vs time graph for the internal space of the housing according to various embodiments;

FIG. 5 shows a cross-sectional view of a disinfection apparatus according to various embodiments;

FIG. 6 shows a screw of the disinfection apparatus of FIG. 5 according to various embodiments;

FIG. 7 shows a cross-sectional view of a disinfection apparatus according to various embodiments;

FIG. 8 shows a perspective front view of the disinfection apparatus of FIG. 7 according to various embodiments;

FIG. 9 shows a perspective back view of the disinfection apparatus of FIG. 7 according to various embodiments;

FIG. 10 shows a top view of the disinfection apparatus of FIG. 7 according to various embodiments;

FIG. 11 shows a bottom view of the disinfection apparatus of FIG. 7 according to various embodiments; and

FIG. 12 shows a right side view of the disinfection apparatus of FIG. 7 according to various embodiments.

### DETAILED DESCRIPTION

Embodiments described below in the context of the apparatus are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural references unless context clearly indicates otherwise. Similarly, the word “or” is intended to include “and” unless the context clearly indicates otherwise.

Various embodiments generally relate to a disinfection apparatus and a method of treating waste. In particular, various embodiments relate to a disinfection apparatus for treating waste collected from a toilet or a lavatory or a livestock farm. Further, various embodiments relate to a portable standalone disinfection apparatus that may be brought to rural places for direct treatment of waste collected in-situ before using the treated waste as fertilizer for agriculture. Various embodiments may also relate to a scale-up industrial disinfection apparatus in a centralized waste treatment facility that may be used for treatment of waste accumulated or collected or consolidated from a community of populated area or a district or a precinct or a settlement or a city or a town or a village etc. so as to convert the waste suitable for use in agriculture. According to various embodiments, waste may refer to the faeces (or solids) collected and separated from the sewage of the toilet or the lavatory, or the livestock farm waste.

Various embodiments seek to provide a disinfection apparatus and a method of treating waste that addresses the above-identified issues. Various embodiments seek to provide a disinfection apparatus and a method that provide easy and fuss-free portable standalone solution for direct treatment of waste in-situ in rural places. Various embodiments may also seek to provide a solution for treatment of waste so as to turn the waste into material, e.g. fertilizer, suitable for use in agriculture.

Various embodiments may be configured to be installed directly to independent toilet or lavatory or livestock farm waste collection point found in rural areas for direct independent treatment of waste. Accordingly, various embodiments may be configured to be portable or easily transportable. Various embodiments may also be configured for use with centralized sewage treatment facilities so as to convert the waste collected at the centralized sewage treatment facilities into material, e.g. fertilizer, suitable for use in agriculture. Various embodiments seek to provide a thermal disinfection apparatus and a method of treating waste via thermal disinfection and/or drying.

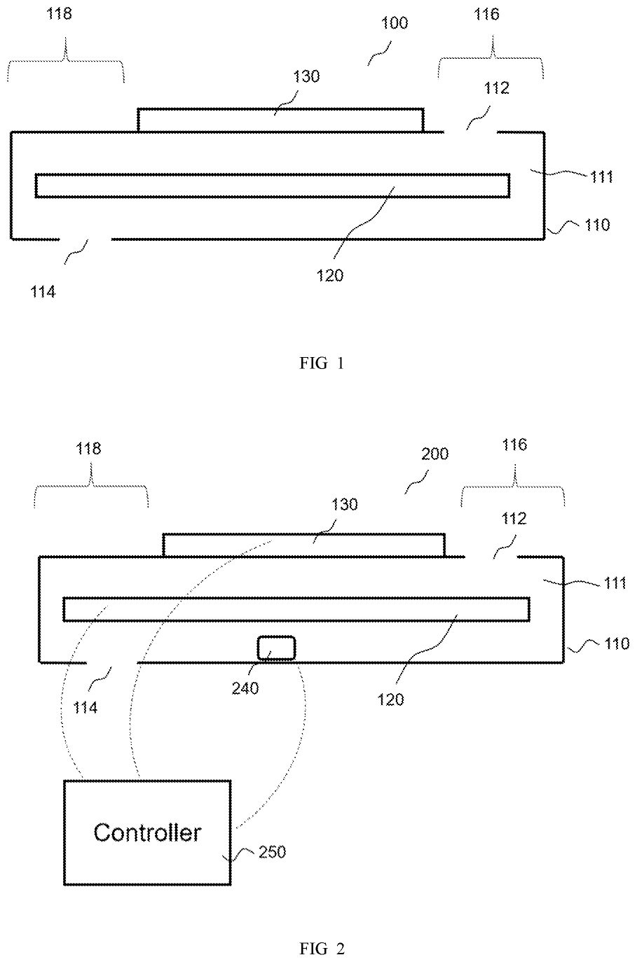

FIG. 1 shows a schematic diagram of a disinfection apparatus 100 for treating waste according to various embodiments. As shown, the disinfection apparatus 100 may include a housing 110 having an inlet 112 and an outlet 114. The housing 110 may be a structure or a cover or a case surrounding an internal space 111. The waste may enter the housing 110 via the inlet 112 such that the waste may be treated within the housing 110. Subsequently, the treated or processed waste may exit the housing 110 via the outlet 114. The disinfection apparatus 100 may further include a conveying mechanism 120 disposed within the housing 110. The conveying mechanism 120 may extend at least substantially along a length of the housing 110 from the inlet 112 of the housing 110 to the outlet 114 of the housing 110. Accordingly, the conveying mechanism 120 may be contained inside the housing 110 and may be disposed such that the extent of the conveying mechanism 120 stretches from the inlet 112 of the housing 110 to the outlet 114 of the housing 110. Hence, the waste which enters the inlet 112 of the housing 110 may be conveyed or transported or carried or moved or transferred by the conveying mechanism 120 to the outlet 114 of the housing 110. Furthermore, the disinfection apparatus 100 may include a heating mechanism 130 configured transfer heat to the housing so as to heat the internal space 111 of the housing 110 to create a heated environment within the housing. Accordingly, the heating mechanism 130 may heat up the housing 110 such that the internal space 111 inside the housing 110 may be heated up by the heated housing 110 for the internal space 111 to become the heated environment. Hence, the waste which is deposited into the housing 110 of the disinfection apparatus 100 may be exposed to the heated environment inside the housing 110 for thermal disinfection and/or drying of the waste.

According to various embodiments, the housing 110 of the disinfection apparatus 100 may be elongated in shape. Accordingly, the housing 110 may be of a long, linear, narrow, or slender shape such as cylindrical, or tube-like, or bar-like, or rod-like shape. The inlet 112 of the housing 110 may be disposed at a longitudinal end portion 116 (or a first longitudinal end portion) of the elongated housing 110 and the outlet 114 of the housing 110 may be disposed at an opposite longitudinal end portion 118 (or a second longitudinal end portion) of the elongated housing 110. Accordingly, the inlet 112 of the housing 110 and the outlet 114 of the housing 110 may be on opposite longitudinal end portions 116, 118 of the elongated housing 110 such that waste which has entered the housing 110 via the inlet 112 has to be conveyed or transported or carried or moved or transferred by the conveying mechanism 120 along a length of the elongated housing 110 from the longitudinal end portion 116 of the elongated housing 110 with the inlet 112 to the opposite longitudinal end portion 118 of the elongated housing 110 with the outlet 114 in order for the waste to exit via the outlet 114 of the housing 110. Hence, the waste may be continuously exposed to the heated environment to undergo thermal disinfection and/or drying within the housing 110 as the waste is conveyed or transported or carried or moved or transferred from the inlet 112 of the housing 110 to the outlet 114 of the housing 110. According to various embodiments, the rate of movement of the waste from the inlet 112 of the housing 110 to the outlet 114 of the housing may be configured such that the waste may be exposed to the heated environment for a pre-determined duration while being conveyed or transported or carried or moved or transferred.

According to various embodiments, the inlet 112 and the outlet 114 of the housing 110 may be facing opposite directions. Accordingly, the inlet 112 may be oriented in a first direction and the outlet 114 may be oriented in a second direction whereby the first direction and the second direction are opposite directions. For example, the inlet 112 may be oriented upwards while the outlet 114 may be oriented downwards when the disinfection apparatus 100 is in use (i.e. in an orientation whereby the disinfection apparatus 100 is installed for use). Hence, the waste may enter the housing 110 of the disinfection apparatus 100 through the inlet 112 via gravity. Similarly, the waste may exit the housing 110 of the disinfection apparatus 100 through the outlet 114 via gravity.

According to various embodiments, the conveying mechanism 120 may include a screw conveyor mechanism, or a bucket conveyor mechanism, or a drag chain conveyor mechanism, or a belt conveyor mechanism, or a wire mesh conveyor mechanism, or a roller conveyor mechanism, or a spiral conveyor mechanism, or any other suitable conveyor mechanism that may convey or transport or carry or move or transfer waste from the first longitudinal end portion 116 of the housing 110 to the second opposite longitudinal end portion 118 of the housing 110.

According to various embodiments, the heating mechanism 130 may include one or more heating elements directly coupled to the housing 110. Accordingly, the one or more heating elements of the heating mechanism 130 may be in direct contact with the housing 110. According to various embodiments, the heating mechanism may include an electric heater that converts electric current to heat. Accordingly, the one or more heating elements may convert electrical energy to heat for heating the housing 110 via conduction of heat. The heated housing 110 may then heat up the internal space 111 within the housing 110 to create the heated environment for thermal disinfection and/or drying of the waste inside the housing 110.

According to various embodiments, the one or more heating elements may be configured to wrap around or surround the housing 110 such that the one or more heating elements may provide a uniform heating around the housing 110 or circumferentially. According to various embodiments, there may be more than one heating elements (or a plurality of heating elements). Accordingly, heating elements may be lined in sequence along the length of the housing 110 to provide a uniform heating along the length of the housing 110 or lengthwise. According to various embodiments, the one or more heating elements may include a band heater, or a heater pad, or a heater plate, or a heating net, or a heater coil, or a heater wire, or a heater rod, or a heater fin, or any combination thereof or other suitable heating elements.

FIG. 2 shows a schematic diagram of a disinfection apparatus 200 for treating waste according to various embodiments. The disinfection apparatus 200 of FIG. 2 contains all the features of the disinfection apparatus 100 of FIG. 1. Accordingly, all features, changes, modifications, and variations that are applicable to the disinfection apparatus 100 of FIG. 1 are also applicable to the disinfection apparatus 200 of FIG. 2. According to various embodiments, the disinfection apparatus 200 of FIG. 2 differs from the disinfection apparatus 100 of FIG. 1 in that the disinfection apparatus 200 of FIG. 2 may include the following additional features.

According to various embodiments, the disinfection apparatus 200 of FIG. 2 may further include one or more temperature sensors 240 disposed and configured to measure a temperature of the internal space 111 of the housing 110. Accordingly, the one or more temperature sensors 240 may provide feedback regarding a temperature of the heated environment within the housing 110. According to various embodiments, the one or more temperature sensors 240 may be disposed at or within the housing 110. According to various embodiments, the one or more temperature sensors 240 may be located or disposed at any point or position at or within the housing 110. According to various embodiments, the temperature sensors 240 may include thermocouple, or resistance temperature detector, or semiconductor-based sensor, or temperature detector with multiple sensing points, or other suitable type of temperature sensing devices.

According to various embodiments, the disinfection apparatus 200 of FIG. 2 may further include a controller 250. The controller 250 may be understood as any kind of a logic implementing entity, which may be special purpose circuitry or a processor executing software stored in a memory, firmware, or any combination thereof. Thus, the controller 250 may be a hard-wired logic circuit or a programmable logic circuit such as a programmable processor (e.g. a Programmable Logic Controller (PLC)), e.g. a microprocessor (e.g. a Complex Instruction Set Computer (CISC) processor or a Reduced Instruction Set Computer (RISC) processor). The controller 250 may also be a processor executing software, e.g. any kind of computer program, e.g. a computer program using a virtual machine code such as e.g. Java. According to various embodiments, the controller 250 may be integrated in the respective apparatus of the various embodiments or may be a separate device connected to the respective apparatus of the various embodiments.

According to various embodiments, the controller 250 may be electrically coupled to the one or more temperature sensors 240, the heating mechanism 130 and the conveying mechanism 120. Accordingly, the controller 250 may be configured to receive signals from the one or more temperature sensors regarding the temperature detected. The controller 250 may also be configured to send instructions to the heating mechanism 130 and the conveying mechanism 120 to operate, or activate, or control, or command the heating mechanism 130 and the conveying mechanism 120 respectively.

According to various embodiments, the controller 250 may be configured to control the heating mechanism 130 based on feedback from the one or more temperature sensors 240 to control the temperature of the internal space 111 of the housing 110 for thermal disinfection and/or drying of the waste. Hence, the controller 250 may be configured to control the heating mechanism 130 based on feedback from the one or more temperature sensors 240 to maintain the heated environment within the housing 110 at a predetermined temperature. Accordingly, the controller 250, the one or more temperature sensors 240 and the heating mechanism 130 may form a closed-loop temperature control system to manage the temperature of the internal space 111 within the housing 110. Hence, the temperature of the internal space 111 within the housing 110 may be maintained or regulated such that the heated environment is at a constant predetermined temperature. According to various embodiments, the predetermined temperature of the heated environment for thermal disinfection and/or drying of the waste may be at least 70° C., or between 70° C. to 200° C., or between 90° C. to 200°, or between 100° C. to 200°, or between 70° C. to 150° C., or between 90° C. to 150°, or between 100° C. to 150°, or between 70° C. to 130° C., or between 90° C. to 130° C., or between 100° C. to 130° C. or between 115° C. to 125° C., or about 120° C. For example, a temperature above 70° C. may already be suitable for thermal disinfection. Further, a temperature above 100° C. may be suitable for both thermal disinfection and drying.

According to various embodiments, the controller 250 may be configured to control the conveying mechanism 120 to repeatedly move in a first operation direction (or first direction) and in a second reverse direction (or second direction) so as to move the waste in a first longitudinal direction and in a second opposite longitudinal direction along the housing based on a predetermined sequence. According to various embodiments, the first longitudinal direction and the second opposite longitudinal direction may be along the length of the housing 110. Accordingly, the conveying mechanism 120 may be controlled to move the waste up and down along the length of the housing 110 based on the predetermined sequence. According to various embodiments, the conveying mechanism 120 may be controlled to move the waste along the housing 110 up to and before the outlet 114 of the housing 110 such that the waste may be kept within the housing 110 without exiting the housing 110. According to various embodiments, when the conveying mechanism 120 is a screw conveyor mechanism, the first operation direction may be a clockwise direction of the screw of the conveying mechanism 120 and the second reverse direction may be a counter-clockwise direction of the screw of the conveying mechanism 120. Accordingly, the screw of the screw conveyor mechanism 120 may be controlled to rotate clockwise and counter-clockwise based on the predetermined sequence so as to move the waste up (or in the first longitudinal direction) and down (or in the second longitudinal direction) along the length of the housing 110. According to various embodiments, the predetermined sequence may include an order of movement of the conveying mechanism 120 in the respective directions and time allocated to each movement. According to various embodiments, an equal amount of time may be allocated to each movement. According to various embodiments, the sequence of movement of the conveying mechanism 120 to move the waste up and down along the length of the housing 110 may repeatedly mix and stir the waste in order to uniformly heat the waste to enhance the thermal disinfection and/or drying process of the waste. According to various embodiments, the predetermined sequence may be performed or conducted within a predetermined period of time. Accordingly, the predetermined sequence may be performed or conducted by the controller 250 during the predetermined period of time. According to various embodiments, at the end of the predetermined sequence and/or at the end of the predetermined period of time, the controller 250 may be configured to control the conveying mechanism to convey or transport or carry or move or transfer the waste along the housing 110 from the longitudinal end portion 116 of the housing 110 with the inlet 112 to the opposite longitudinal end portion 118 of the housing 110 with the outlet 114 such that the waste may exit the housing 110 through the outlet 114. According to various embodiments, the predetermined period of time may be approximately between 60 minutes to 120 minutes (1 hour to 2 hours), or about 120 minutes (2 hours). According to various embodiments, the controller 250 may be configured to control the heating mechanism to heat the internal space 111 within the housing 110 so as to maintain or regulate the temperature of the internal space 111 within the housing 110 at the predetermined temperature of the heated environment while the conveying mechanism 120 is in operation.

According to various other embodiments, the controller 250 may be configured to activate the conveying mechanism 120 after the internal space 111 of the housing 110 is maintained at the predetermined temperature for the predetermined period of time. Accordingly, when the waste is deposited into the housing 110 of the disinfection apparatus 100 through the inlet 112, the waste may be kept or maintained or retained stationary inside the longitudinal end portion 116 of the housing 110 with the inlet 112. The waste may be maintained inside the longitudinal end portion 116 of the housing 110 with the inlet 112 for the predetermined period of time in the heated environment to undergo thermal disinfection and/or drying, before the conveying mechanism 120 is activated by the controller 250 to convey or transport or carry or move or transfer the waste from the longitudinal end portion 116 of the housing 110 with the inlet 112 to the opposite longitudinal end portion 118 of the housing 110 with the outlet 114 such that the waste may exit the housing 110 through the outlet 114. According to various embodiments, the predetermined period of time may be approximately between 60 minutes to 120 minutes, or about 120 minutes.

According to various embodiments, the controller 250 may be configured to maintain the heated environment within the housing 110 at the predetermined temperature while the conveying mechanism 120 is in operation. Accordingly, after the conveying mechanism 120 is activated by the controller 250 to convey or transport or carry or move or transfer the waste, the controller 250 may continue to control the temperature of the internal space 111 inside the housing 110 via controlling the heating mechanism 130 based on feedback from the one or more temperature sensors 240 so as to maintain the internal space 111 at the predetermined temperature to maintain the heated environment for further thermal disinfection and/or drying of the waste as the waste is in motion.

According to various embodiments, the controller 250 may be configured to control the heating mechanism 130 and the conveying mechanism 120 so as to disinfect and/or dry the waste deposited, supplied or fed into the housing 110 of the disinfection apparatus 100. Accordingly, the controller 250 may be configured to control the heating mechanism 130 and the conveying mechanism 120 to remove pathogen, such as total coliform bacteria etc., from the waste. Further, the controller 250 may be configured to control the heating mechanism 130 and the conveying mechanism 120 to reduce moisture content of the waste from 84% to 99% of total mass to 41% to 52% of total mass (as measured based on the standard test methods for moisture—ASTM D 2974-87).

According to various embodiments, the controller 250 may be programmable such that a user may set the predetermined temperature for the heated environment as well as set the duration before the conveying mechanism 120 is activated. Accordingly to various embodiments, the controller 250 may include a user interface for receiving inputs from the user. FIG. 3 shows a method 300 of treating waste according to various embodiments.

According to various embodiments, the method 300 may optionally use the disinfection apparatus as described herein. According to various embodiments, the method 300 of treating waste may include, at 302, providing the waste in a heated environment for thermal disinfection and/or drying within a housing of a disinfection apparatus. The disinfection apparatus may be according to the various embodiments as described herein. Accordingly, the internal space of the housing may be at the predetermined temperature forming the heated environment when the waste is deposited into the housing. According to various embodiments, the method 300 of treating waste may further include, at 304, conveying the waste in the heated environment, via a conveying mechanism of the disinfection apparatus, along the length of the housing of the disinfection apparatus. Accordingly, the waste may be continuously exposed to the heated environment for further thermal disinfection and/or drying within the housing as the waste is conveyed or transported or carried or moved or transferred along the length of the housing. According to various embodiments, the housing may include an inlet and an outlet. Accordingly, the waste may be conveyed or transported or carried or moved or transferred along the length of the housing from the inlet of the housing to the outlet of the housing.

According to various embodiments, the method 300 of treating waste may include heating, via a heating mechanism of the disinfection apparatus, the housing so as to heat the internal space of the housing to create the heated environment for thermal disinfection and/or drying of the waste within the housing. Accordingly, the housing may be heated by the heating mechanism prior to the deposition of the waste to bring up a temperature of the internal space of the housing to the predetermined temperature so as to create the heated environment for thermal disinfection and/or drying of the waste. Upon reaching the predetermined temperature, the housing may continue to be heated by the heating mechanism so as to maintain the internal space of the housing at the predetermined temperature. Hence, the heating of the housing may include a pre-heating phase to bring up the temperature of the internal space of the housing to form the heated environment and a subsequent heating phase to maintain the temperature of the heated environment. According to various embodiments, the waste is deposited into the housing after the heated environment for thermal disinfection and/or drying is formed inside the housing.

According to various embodiments, the predetermined temperature of the heated environment for thermal disinfection and/or drying of the waste may be at least 70° C., or between 70° C. to 200° C., or between 90° C. to 200°, or between 100° C. to 200°, or between 70° C. to 150° C., or between 90° C. to 150°, or between 100° C. to 150°, or between 70° C. to 130° C., or between 90° C. to 130° C., or between 100° C. to 130° C., or between 115° C. to 125° C., or about 120° C. For example, a temperature above 70° C. may already be suitable for thermal disinfection. Further, a temperature above 100° C. may be suitable for both thermal disinfection and drying.

According to various embodiments, the method 300 of treating waste may include holding the waste in the heated environment within the housing for a predetermined period of time before conveying the waste along the length of the housing. Accordingly, after the waste is deposited into the housing of the disinfection apparatus, the waste is kept inside the housing at the longitudinal end portion of the housing near the inlet for the predetermined period of time to undergo thermal disinfection and/or drying before the waste is conveyed or transported or carried or moved or transferred (i.e. at step 304) along the length of the housing from the inlet of the housing to the outlet of the housing. According to various embodiments, the predetermined period of time may be approximately between 60 minutes to 120 minutes, or about 120 minutes.

According to various embodiments, instead of holding the waste in the heated environment within the housing for a predetermined period of time before conveying the waste to the outlet of the housing, the conveying of the waste in 304 of the method 300 may include repeatedly conveying or transporting or carrying or moving or transferring the waste in a first longitudinal direction and in a second opposite longitudinal direction along the housing without discharging the waste at the outlet of the housing based on a predetermined sequence or for a predetermined period of time. Accordingly, the waste may be moved up (in the first longitudinal direction) and down (in the second opposite longitudinal direction) along the housing based on the predetermined sequence or for the predetermined period of time. According to various embodiments, the method 300 may further include heating the housing so as to maintain the heated environment within the housing while the waste is being moved up and down along the housing based on the predetermined sequence or for the predetermined period of time

According to various embodiments, the conveying of the waste in 304 of the method 300 may include conveying the waste from the inlet region of the housing to the outlet region of the housing to discharge the waste from the housing via the outlet such that the time taken to convey the waste from the inlet region of the housing to the outlet region of the housing is between 25 minutes to 120 minutes, or between 25 minutes to 75 minutes, or between 30 minutes to 60 minutes. Accordingly, it may take between 25 minutes to 120 minutes, or between 25 minutes to 75 minutes, or between 30 minutes to 60 minutes to convey or transport or carry or move or transfer the waste from the longitudinal end portion of the housing with the inlet to the opposite longitudinal end portion of the housing with the outlet for discharging the waste through the outlet of the housing. According to various embodiments, while the waste is being conveyed or transported or carried or moved or transferred along the length of the housing from the inlet of the housing to the outlet of the housing, the heated environment within the housing may be maintain to subject the waste to further thermal disinfection and/or drying during the 25 minutes to 120 minutes, or 25 minutes to 75 minutes, or 30 minutes to 60 minutes of conveying time. According to various embodiments, the method 300 may further include heating the housing so as to maintain the heated environment within the housing as the waste is being conveyed along the length of the housing from the inlet region of the housing to the outlet region of the housing for discharging the waste through the outlet of the housing.

According to various embodiments, the method 300 may disinfect and/or dry the waste deposited, supplied or fed into the housing of the disinfection apparatus. Accordingly, the method 300 of the various embodiments may remove pathogen, such as total coliform bacteria etc., from the waste. Further, the method 300 of the various embodiments may also reduce the moisture content of the waste from 84% to 99% of total mass to 41% to 52% of total mass (as measured based on the standard test methods for moisture—ASTM D 2974-87).

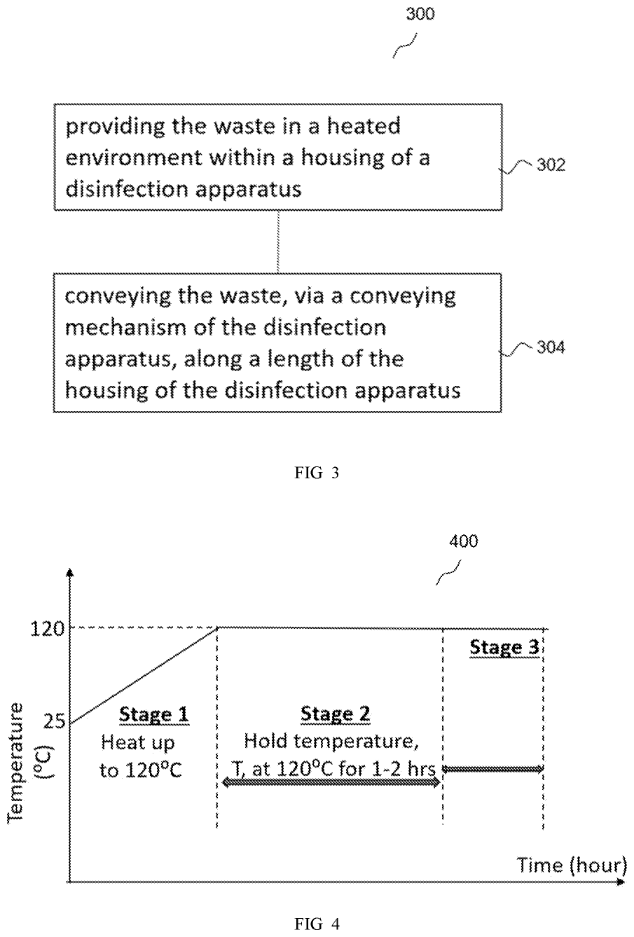

FIG. 4 shows a temperature vs time graph 400 for the internal space of the housing according to various embodiments. As shown, at stage 1, the internal space of the housing may be heated up to the predetermined temperature, which may be about 120° C., from room temperature (for example, between 25° C.-35° C.), to form the heated environment for thermal disinfection and/or drying of the waste. At stage 2, the temperature, T, of the internal space of the housing may be held at the predetermined temperature for between 60 minutes to 120 minutes (1 hour to 2 hours), or about 120 minutes (2 hours) so as to maintain the temperature of the heated environment for thermal disinfection and/or drying of the waste within the housing. During this stage, the waste may be deposited into the housing of the disinfection apparatus via the inlet at the beginning of the stage and be kept in the heated environment throughout the approximately between 60 minutes to 120 minutes, or about 120 minutes duration. During this stage, the waste may be kept stationary throughout the entire duration. The waste may also be conveyed up and down along the housing of the disinfection apparatus throughout the entire duration to stir and mix the waste for uniform heating of the waste to enhance thermal disinfection and/or drying. Further, the waste may also be kept stationary during part of the entire duration and conveyed up and down along the housing of the disinfection apparatus during another part of the entire duration. At stage 3, the temperature, T, of the internal space of the housing may continue to be held at the predetermined temperature while the conveying mechanism may be activated to convey or transport or carry or move or transfer the waste from the longitudinal end portion of the housing with the inlet to the opposite longitudinal end portion of the housing with the outlet for discharging the waste such that the waste is subjected to further thermal disinfection and/or drying during conveyance.

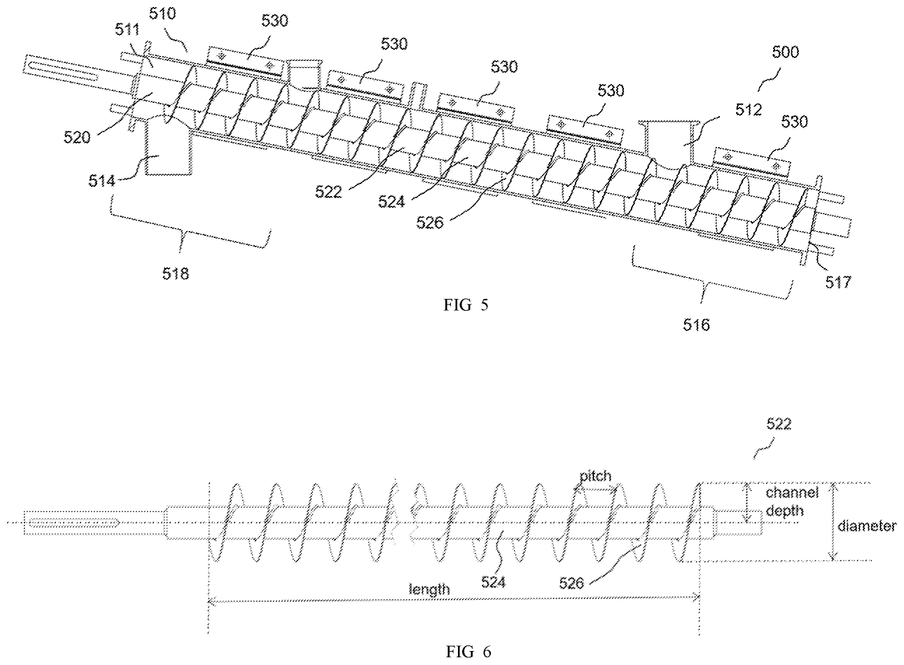

FIG. 5 shows a cross-sectional view of a disinfection apparatus 500 according to various embodiments. The disinfection apparatus 500 of FIG. 5 contains all the features of the disinfection apparatus 100 of FIG. 1. Accordingly, all features, changes, modifications, and variations that are applicable to the disinfection apparatus 100 of FIG. 1 are also applicable to the disinfection apparatus 500 of FIG. 5. The disinfection apparatus 500 of FIG. 5 may also contain all the additional features of the disinfection apparatus 200 of FIG. 2. Accordingly, all features, changes, modifications, and variations that are applicable to the disinfection apparatus 200 of FIG. 2 may also be applicable to the disinfection apparatus 500 of FIG. 5. According to various embodiments, the disinfection apparatus 500 of FIG. 5 differs from the disinfection apparatus 100 of FIG. 1 as well as the disinfection apparatus 200 of FIG. 2 in that the disinfection apparatus 500 of FIG. 5 may include the following additional limitations and/or features.

As shown, the housing of the disinfection apparatus 500 of FIG. 5 may be a cylindrical tubular housing 510 having an inlet duct 512 and an outlet duct 514. The conveying mechanism of the disinfection apparatus 500 of FIG. 5 may be screw conveyor mechanism 520 disposed inside the cylindrical tubular housing 510. The screw conveyor mechanism 520 may extend at least substantially along a length of the cylindrical tubular housing 510 from the inlet duct 512 to the outlet duct 514. As shown, the screw conveyor mechanism 520 may extend along the entire length of the cylindrical tubular housing 510. Further, the heating element of the heating mechanism of the disinfection apparatus 500 of FIG. 5 may be a plurality of band heaters 530 for clamping around the cylindrical tubular housing 510 along the length of the cylindrical tubular housing 510. As shown, the disinfection apparatus 500 of FIG. 5 may include five band heaters 530 spread along the length of the cylindrical tubular housing 510. Accordingly, the plurality of band heaters 530 may be configured to transfer heat to the cylindrical tubular housing 510 so as to heat the internal space 511 of the cylindrical tubular housing 510 to create a heated environment for thermal disinfection and/or drying of the waste within the cylindrical tubular housing 510.

According to various embodiments, the inlet duct 512 and the outlet duct 514 may be projecting away from the cylindrical surface of the cylindrical tubular housing 510 in opposite directions. Accordingly, the inlet duct 512 may be projecting away from a first side of the cylindrical surface of the cylindrical tubular housing 510 and the outlet duct 514 may be projecting away from a second side of the cylindrical surface of the cylindrical tubular housing 510, whereby the first side and the second side may be opposite sides of the cylindrical surface of the cylindrical tubular housing 510. According to various embodiments, each of the inlet duct 512 and the outlet duct 514 may form an angle of approximately between 15° to 90°, or between 75° to 80°, or about 78°, with respect to the cylindrical surface the cylindrical tubular housing 510. Hence an axis of the inlet duct 512 may form an angle of approximately between 15° to 90°, or between 75° to 80°, or about 78°, with respect to a longitudinal axis of the cylindrical tubular housing 510. Similarly, an axis of the outlet duct 514 may form an angle of approximately between 15° to 90°, or between 75° to 80°, or about 78°, with respect to a longitudinal axis of the cylindrical tubular housing 510. Accordingly, when the disinfection apparatus 500 is installed for use, the disinfection apparatus 500 may be installed at an angle of approximately between 0° to 75°, or between 10° to 15° or about 12° with respect to the ground (or horizontal) in order for the the inlet duct 512 and the outlet duct 514 to be in a vertical orientation with respect to the ground. Accordingly, with the disinfection apparatus 500 installed in an inclined orientation, waste deposited into the cylindrical tubular housing 510 through the inlet duct 512, via gravity, may be accumulated at the longitudinal end portion 516 (or first longitudinal end portion) of the cylindrical tubular housing 510.

As shown, while the inlet duct 512 is at the longitudinal end portion 516 of the cylindrical tubular housing 510, the inlet duct 512 is not at an extreme end 517 of the longitudinal end portion 516 of the cylindrical tubular housing 510. Rather, the inlet duct 512 is located at the longitudinal end portion 516 of the cylindrical tubular housing 510 with some distance from the extreme end 517 of the longitudinal end portion 516 of the cylindrical tubular housing 510. Accordingly, the configuration may allow waste deposited into the cylindrical tubular housing 510 to be accumulated between the extreme end 517 of the cylindrical tubular housing 510 and the inlet duct 512. As shown, a band heater 530 may be clamped around a segment of the cylindrical tubular housing 510 between the extreme end 517 of the cylindrical tubular housing 510 and the inlet duct 512. In this manner, the band heater 530 may ensure that the segment of the longitudinal end portion 516 of the cylindrical tubular housing 510 between the extreme end 517 of the cylindrical tubular housing 510 and the inlet duct 512 may be maintained at the predetermined temperature of the heated environment for thermal disinfection and/or drying of the waste contained therein.

FIG. 6 shows a screw 522 of the screw conveyor mechanism 520 of the disinfection apparatus 500 of FIG. 5 according to various embodiments. As shown, the screw 522 may include a shaft 524 with a helical blade 526 (or a spiral blade). According to various embodiments, a motor may be coupled to the shaft 524 of the screw 522 so as to drive a rotation of the shaft 524 in order to rotate the screw 522 about the longitudinal axis of the screw 522. According to various embodiments, the screw 522 of the screw conveyor mechanism 520 may extend at least substantially along the length of the cylindrical tubular housing 510 from the inlet duct 512 of the cylindrical tubular housing 510 to the outlet duct 514 of the cylindrical tubular housing 510 such that the screw 522 may be rotated to convey or transport or carry or move or transfer the waste received through the inlet duct 512 to the outlet duct 514. As shown in FIG. 5, the screw 522 of the screw conveyor mechanism 520 may extend through the entire length of the cylindrical tubular housing 510. Accordingly, the screw 522 may also be effective in conveying or transporting or carrying or moving or transferring the waste accumulated near the extreme end 517 of the longitudinal end portion 516 of the cylindrical tubular housing 510 towards the outlet duct 514.

According to various embodiments, the screw 522 may be configured in terms of a pitch of the helical blade 526 of the screw 522, a length of the helical blade 526 of the screw 522, and a rotational speed of the screw 522 to move the waste from the inlet duct region of the cylindrical tubular housing 510 to the outlet duct region of the cylindrical tubular housing 510 such that a time taken for the waste to move from inlet duct region of the cylindrical tubular housing 510 to the outlet duct region of the cylindrical tubular housing 510 is between 25 minutes to 120 minutes, or between 25 minutes to 75 minutes, or between 30 minutes to 60 minutes. Accordingly, it may take between 25 minutes to 120 minutes, or between 25 minutes to 75 minutes, or between 30 minutes to 60 minutes to convey or transport or carry or move or transfer the waste from the longitudinal end portion 516 of the housing 510 with the inlet duct 512 to the opposite longitudinal end portion 518 of the housing 510 with the outlet duct 514.

According to various embodiments, a profile of the helical blade 526 of the screw 522 may include a longitudinal length to lateral diameter ratio of approximately between 10.5 to 14.5, a longitudinal length to pitch ratio of approximately between 17.5 to 25.5, and a channel depth to shaft's radius ratio of approximately between 2.5 to 3.6. Further, the shaft 524 of the screw 522 may be configured to have a rotational speed of about 10 rounds per minute (RPM).

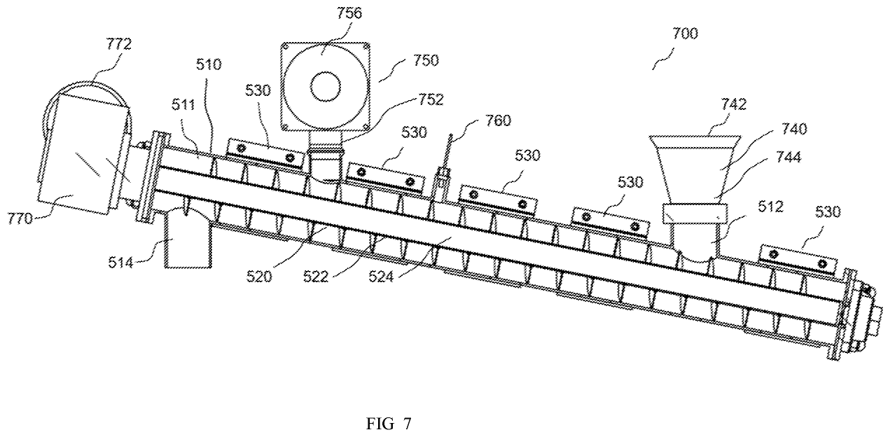

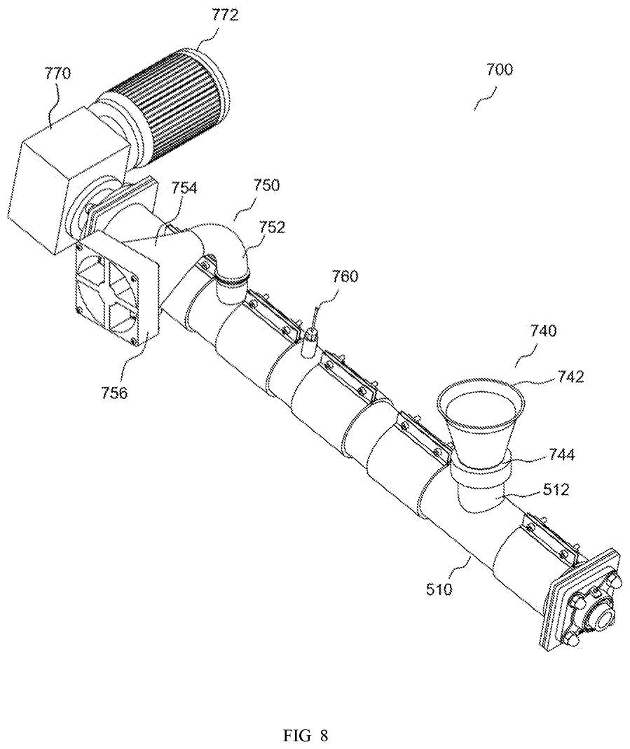

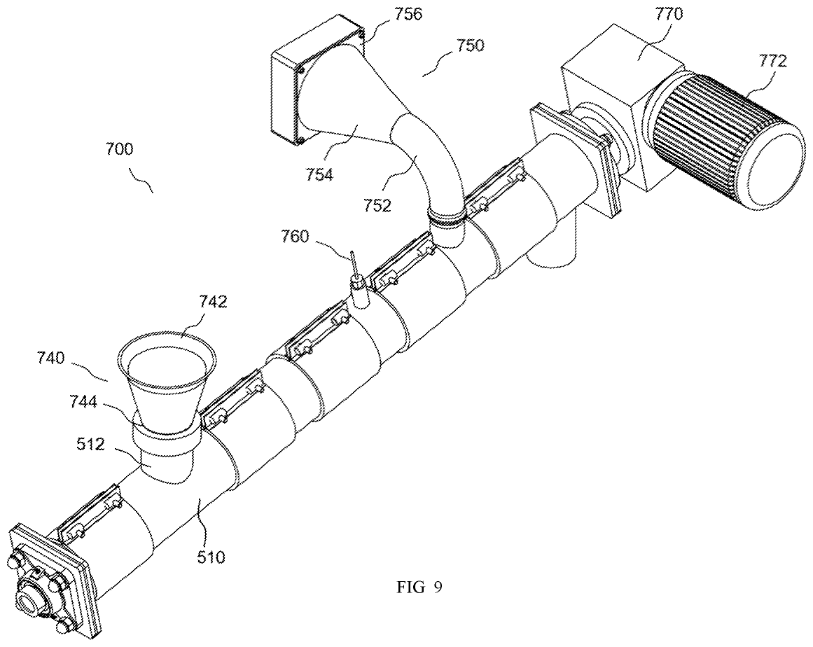

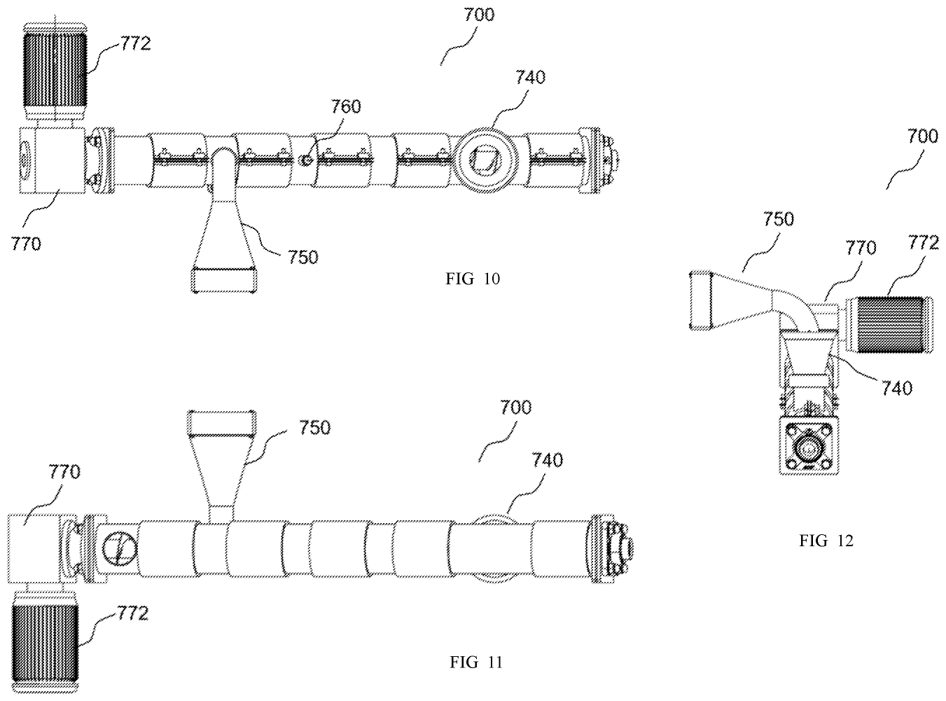

FIG. 7 shows a cross-sectional view of a disinfection apparatus 700 according to various embodiments. FIG. 8 to FIG. 12 shows a perspective front view, a perspective back view, a top view, a bottom view, and a right side view respectively of the disinfection apparatus 700 of FIG. 7 according to various embodiments. The disinfection apparatus 700 of FIG. 7 to FIG. 12 contains all the features of the disinfection apparatus 100 of FIG. 1, the disinfection apparatus 200 of FIG. 2, and the disinfection apparatus 500 of FIG. 5. Accordingly, all features, changes, modifications, and variations that are applicable to the disinfection apparatus 100 of FIG. 1, the disinfection apparatus 200 of FIG. 2, and the disinfection apparatus 500 of FIG. 5 are also applicable to the disinfection apparatus 700 of FIG. 7 to FIG. 12. According to various embodiments, the disinfection apparatus 700 of FIG. 7 to FIG. 12 differs from the disinfection apparatus 100 of FIG. 1, the disinfection apparatus 200 of FIG. 2, and/or the disinfection apparatus 500 of FIG. 5 in that the disinfection apparatus 700 of FIG. 7 to FIG. 12 may include the following additional limitations and/or features.

According to various embodiments, the disinfection apparatus 700 of FIG. 7 to FIG. 12 may further include a hopper 740 coupled to the inlet duct 512 of the cylindrical tubular housing 510. The hopper 740 may be a funnel-shaped structure or a trapezium-shaped structure which may be configured to have a broader or wider opening 742 at a top for receiving the waste and a narrower opening 744 at a bottom for dispensing the waste into the inlet duct 512 of the housing 510. Accordingly, the hopper 740 may allow easy deposition of the waste whereby broader or wider opening 742 at a top of the hopper 740 may minimize the waste from spillage.

According to various embodiments, the disinfection apparatus 700 of FIG. 7 to FIG. 12 may further include an air vent 750 at the cylindrical tubular housing 510. The air vent 750 may be located at the opposite longitudinal end portion 518 of the cylindrical tubular housing 510. According to various embodiments, the air vent 750 may be configured to ventilate the internal space 511 of the cylindrical tubular housing 510 such that moisture vaporized from the waste in the heated environment of the cylindrical tubular housing 510 during thermal disinfection and/or drying of the waste inside the cylindrical tubular housing 510 may escape the internal space 511 of the cylindrical tubular housing 510 as vapor via the air vent 750. According to various embodiments the air vent 750 may include an air duct 752 with a flared opening 754. According to various embodiments a fan 756 may be coupled to the flared opening 754 of the air duct 752 to facilitate extraction of vapor from the internal space 511 of the cylindrical tubular housing 510.

According to various embodiments, the one or more temperature sensors of the disinfection apparatus 700 of FIG. 7 to FIG. 12 may be a thermocouple 760 inserted into the internal space 511 of the cylindrical tubular housing 510 via an opening in the cylindrical tubular housing 510 as shown in FIG. 7. The thermocouple 760 may measure a temperature of the internal space 511 of the cylindrical tubular housing 510 and provide feedback regarding the temperature of the heated environment such that the plurality of band heaters 530 may be controlled to regulate or maintain the temperature of the heated environment at the predetermined temperature. According to various embodiments, the one or more temperature sensors may be located in or at any locations along the cylindrical tubular housing 510. Accordingly, the thermocouple 760 may include multiple sensing points and may be located in or at any locations along the cylindrical tubular housing 510.

According to various embodiments, the disinfection apparatus 700 of FIG. 7 to FIG. 12 may include a gear box 770 connecting the shaft 524 of the screw 522 of the screw conveyor mechanism 520 to a motor 772 for driving the rotation of the screw 522. Accordingly, the motor 772 may be operated to rotate the screw 522 of the screw conveyor mechanism 520 for conveying or transporting or carrying or moving or transferring the waste along the cylindrical tubular housing 510.

The following examples pertain to various embodiments.

Example 1 is a disinfection apparatus for treating waste including:
a housing having an inlet and an outlet;
a conveying mechanism which is disposed within the housing and which extends at least substantially along a length of the housing from the inlet to the outlet; and
a heating mechanism configured to transfer heat to the housing so as to heat an internal space of the housing to create a heated environment within the housing

In Example 2, the subject matter of Example 1 may optionally include that the housing may be elongated in shape, and wherein the inlet may be disposed at a longitudinal end portion of the elongated housing and the outlet may be disposed at an opposite longitudinal end portion of the elongated housing.

In Example 3, the subject matter of Example 2 may optionally include that the inlet of the housing and the outlet of the housing may be facing opposite directions.

In Example 4, the subject matter of any one of Examples 1 to 3 may optionally include that the conveying mechanism may include a screw conveyor mechanism, or a bucket conveyor mechanism, or a drag chain conveyor mechanism, or a belt conveyor mechanism, or a wire mesh conveyor mechanism, or a roller conveyor mechanism, or a spiral conveyor mechanism.

In Example 5, the subject matter of any one of Examples 1 to 3 may optionally include that the conveying mechanism may include a screw conveyor mechanism, the screw conveyor mechanism may include a screw having a shaft with a helical blade or a spiral blade.

In Example 6, the subject matter of Example 5 may optionally include that the screw of the screw conveyor mechanism may extend at least substantially along the length of the housing from the inlet to the outlet.

In Example 7, the subject matter of Example 5 or 6 may optionally include that the screw may be configured in terms of a pitch of the helical blade of the screw, a length of the helical blade of the screw and a rotational speed of the screw. Further, a time taken for the waste to move from an inlet region of the housing to an outlet region of the housing may be between 25 minutes to 120 minutes.

In Example 8, the subject matter of any one of Examples 1 to 7 may optionally include that the heating mechanism may include one or more heating elements directly coupled to the housing.

In Example 9, the subject matter of claim 8 may optionally include that the one or more heating elements may include a band heater, or a heater pad, or a heater plate, or a heating net, or a heater coil, or a heater wire, or a heater rod, or a heater fin, or any combination thereof.

In Example 10, the subject matter of any one of Examples 1 to 9 may optionally include an air vent at the housing.

In Example 11, the subject matter of any one of Examples 1 to 10 may optionally include a hopper coupled to the inlet of the housing.

In Example 12, the subject matter of claim 11 may optionally include that the hopper may include a funnel-shaped structure, or a trapezium-shaped structure.

In Example 13, the subject matter of any one of Examples 1 to 12 may optionally further include:
one or more temperature sensors configured to measure a temperature of the internal space of the housing; and
a controller electrically coupled to the one or more temperature sensors, the heating mechanism and the conveying mechanism.

In Example 14, the subject matter of Example 13 may optionally include that the controller may be configured to control the heating mechanism based on feedback from the one or more temperature sensors to control the temperature of the internal space of the housing.

In Example 15, the subject matter of Example 14 may optionally include that the controller may be configured to control the heating mechanism to maintain the temperature of the internal space of the housing at a predetermined temperature to maintain the heated environment within the housing.

In Example 16, the subject matter of Example 14 or 15 may optionally include that the controller may be configured to control the conveying mechanism to repeatedly move in a first direction and in a second opposite direction based on a predetermined sequence.

In Example 17, the subject matter of Example 15 may optionally include that the controller may be configured to activate the conveying mechanism after the internal space of the housing is maintained at the predetermined temperature for a predetermined period of time.

In Example 18, the subject matter of Example 16 or 17 may optionally include that the controller may be configured to maintain the internal space of the housing at the predetermined temperature while the conveying mechanism is in operation.

Example 19 is a method of treating waste, optionally using a disinfection apparatus according to any one of Examples 1 to 18, the method including:
providing the waste in a heated environment within a housing of a disinfection apparatus; and
conveying the waste in the heated environment, via a conveying mechanism of the disinfection apparatus, along a length of the housing of the disinfection apparatus.

In Example 20, the subject matter of Example 19 may optionally include heating, via a heating mechanism of the disinfection apparatus, the housing so as to heat an internal space of the housing to create the heated environment within the housing.

In Example 21, the subject matter of Example 19 or 20 may optionally include that the heated environment is at a temperature of at least 70° C.

In Example 22, the subject matter of any one of Examples 19 to 21 may optionally include holding the waste in the heated environment within the housing for a predetermined period of time before conveying the waste along the length of the housing.

In Example 23, the subject matter of any one of Examples 19 to 21 may optionally include that the conveying the waste includes repeatedly moving the waste in a first longitudinal direction and in a second opposite longitudinal direction along the housing based on a predetermined sequence or a predetermined period of time. The predetermined period of time may be between 60 minutes to 120 minutes.

In Example 24, the subject matter of any one of Example 19 to 23 may optionally include that conveying the waste may include conveying the waste along a length of the housing from an inlet region of the housing to an outlet region of the housing to discharge the waste from the housing via the outlet. Further, the time taken to convey the waste from the inlet region of the housing to the outlet region of the housing may be between 25 minutes to 120 minutes.

In Example 25, the subject matter of any one of Example 24 may optionally include heating the housing so as to maintain the heated environment within the housing as the waste is being conveyed along the length of the housing from the inlet region of the housing to the outlet region of the housing for discharging the waste through the outlet of the housing.

Various embodiments have provided a disinfection apparatus that addresses the various issues identified earlier. For example, various embodiments have provided a portable standalone disinfection apparatus that may be brought to rural places for direct treatment of waste collected in-situ before using the treated waste for agriculture. Various embodiments have also provided a disinfection apparatus and a method of treating waste that provide easy and fuss-free portable standalone solution for direct treatment of waste in-situ in rural places. Various embodiments have also provided a disinfection apparatus that may be configured to be installed directly to independent toilet or lavatory or livestock farm waste collection point found in rural areas for direct independent treatment of waste via thermal disinfection and/or drying. Various embodiments, have also provided a disinfection apparatus for thermal disinfection and/or drying of waste that may be portable or easily transportable.

While the invention has been particularly shown and described with reference to specific embodiments, it should be understood by those skilled in the art that various changes, modification, variation in form and detail may be made therein without departing from the scope of the invention as defined by the appended claims. The scope of the invention is thus indicated by the appended claims and all changes which come within the meaning and range of equivalency of the claims are therefore intended to be embraced.

1. A disinfection apparatus for treating waste comprising:
a housing having an inlet and an outlet;
a conveying mechanism which is disposed within the housing and which extends at least substantially along a length of the housing from the inlet to the outlet; and
a heating mechanism configured to transfer heat to the housing so as to heat an internal space of the housing to create a heated environment within the housing.

2. The disinfection apparatus as claimed in claim 1, wherein the housing is elongated in shape, and wherein the inlet is disposed at a longitudinal end portion of the elongated housing and the outlet is disposed at an opposite longitudinal end portion of the elongated housing.

3. The disinfection apparatus as claimed in claim 2, wherein the inlet of the housing and the outlet of the housing are facing opposite directions.

4. The disinfection apparatus as claimed in claim 1, wherein the conveying mechanism comprises a screw conveyor mechanism, or a bucket conveyor mechanism, or a drag chain conveyor mechanism, or a belt conveyor mechanism, or a wire mesh conveyor mechanism, or a roller conveyor mechanism, or a spiral conveyor mechanism.

5. The disinfection apparatus as claimed in claim 1, wherein the conveying mechanism comprises a screw conveyor mechanism, the screw conveyor mechanism comprising a screw having a shaft with a helical blade or a spiral blade.

6. The disinfection apparatus as claimed in claim 5, wherein the screw of the screw conveyor mechanism extends at least substantially along the length of the housing from the inlet to the outlet.

7. The disinfection apparatus as claimed in claim 5, wherein the screw is configured in terms of a pitch of the helical blade of the screw, a length of the helical blade of the screw and a rotational speed of the screw.

8. The disinfection apparatus as claimed in claim 1, wherein the heating mechanism comprises one or more heating elements directly coupled to the housing.

9. The disinfection apparatus as claimed in claim 8, wherein the one or more heating elements comprises a band heater, or a heater pad, or a heater plate, or a heating net, or a heater coil, or a heater wire, or a heater rod, or a heater fin, or any combination thereof.

10. The disinfection apparatus as claimed in claim 1, further comprising an air vent at the housing.

11. The disinfection apparatus as claimed in claim 1, further comprising a hopper coupled to the inlet of the housing.

12. The disinfection apparatus as claimed in claim 11, wherein the hopper comprises a funnel-shaped structure, or a trapezium-shaped structure.

13. The disinfection apparatus as claimed in claim 1, further comprising:
one or more temperature sensors configured to measure a temperature of the internal space of the housing; and
a controller electrically coupled to the one or more temperature sensors, the heating mechanism and the conveying mechanism.

14. The disinfection apparatus as claimed in claim 13, wherein the controller is configured to control the heating mechanism based on feedback from the one or more temperature sensors to control the temperature of the internal space of the housing.

15. The disinfection apparatus as claimed in claim 14, wherein the controller is configured to control the heating mechanism to maintain the temperature of the internal space of the housing at a predetermined temperature to maintain the heated environment within the housing.

16. The disinfection apparatus as claimed in claim 14 or 15, wherein the controller is configured to control the conveying mechanism to repeatedly move in a first direction and in a second opposite direction based on a predetermined sequence.

17. The disinfection apparatus as claimed in claim 15, wherein the controller is configured to activate the conveying mechanism after the internal space of the housing is maintained at the predetermined temperature for a predetermined period of time.

18. The disinfection apparatus as claimed in claim 16 or 17, wherein the controller is configured to maintain the internal space of the housing at the predetermined temperature while the conveying mechanism is in operation.

19. A method of treating waste, optionally using a disinfection apparatus according to claim 1, the method comprising:
providing the waste in a heated environment within a housing of a disinfection apparatus; and
conveying the waste in the heated environment, via a conveying mechanism of the disinfection apparatus, along a length of the housing of the disinfection apparatus.

20. The method as claimed in claim 19, further comprising heating, via a heating mechanism of the disinfection apparatus, the housing so as to heat an internal space of the housing to create the heated environment within the housing.

21. The method as claimed in claim 19, wherein the heated environment is at a temperature of at least 70° C.

22. The method as claimed in claim 19, further comprising holding the waste in the heated environment within the housing for a predetermined period of time before conveying the waste along the length of the housing.

23. The method as claimed in claim 19, wherein conveying the waste comprises repeatedly moving the waste in a first longitudinal direction and in a second opposite longitudinal direction along the housing based on a predetermined sequence or a predetermined period of time.

24. The method as claimed in claim 19, wherein conveying the waste comprises conveying the waste along a length of the housing from an inlet region of the housing to an outlet region of the housing to discharge the waste from the housing via the outlet.

25. The method as claimed in claim 24, further comprising heating the housing so as to maintain the heated environment within the housing as the waste is being conveyed along the length of the housing from the inlet region of the housing to the outlet region of the housing for discharging the waste through the outlet of the housing.
